(19) United States
(12) Patent Application Publication
(10) Pub. No.: US 2004/0037737 A1
(43) Pub. Date: Feb. 26, 2004

(54) Method of and equipment for washing, disinfecting and/or sterilizing health care devices

(76) Inventors: Jacobus T Marais, Pretoria (ZA); Suha Rawhani, Johannesburg (ZA)

Correspondence Address: NIXON & VANDERHYE, PC, 1100 N GLEBE ROAD, 8TH FLOOR, ARLINGTON, VA, 22201-4714, US

(21) Appl. No.: 10/332,350

(22) PCT Filed: Jul. 9, 2001
(86) PCT No.: PCT/ZA01/00090

(30) Foreign Application Priority Data
Jul. 7, 2000 (ZA) 20001155

(51) Int. Cl. A61L 2/18

(52) U.S. Cl. 422/28; 422/292

(57) ABSTRACT

The invention provides a method for automatically washing, disinfecting and/or sterilizing health care equipment and/or cooking and catering utensils. The method includes the steps of placing the equipment to be washed in an enclosure; introducing a first electrochemically activated aqueous solution into the enclosure; and either sequentially or simultaneously introducing a second electrochemically activated aqueous solution into the enclosure. The first solution is characterised therein that it has dispersing or surfactant characteristics for at least partially dispersing a biofilm, pathogenic microorganisms, contamination or the like. The second solution is characterised therein that it has biocidal characteristics for killing microorganisms and disinfecting and/or sterilizing the equipment. The invention also extends to an apparatus for use in the above method.

### TECHNICAL FIELD

This invention relates to a method of and equipment for washing, disinfecting and/or sterilizing health care devices, including medical, dental or veterinary equipment, as well as cooking and catering utensils. More particularly, but not exclusively, the invention relates to a method of and equipment for automatically washing, disinfecting and/or sterilizing health care devices for use in dentistry.

### BACKGROUND ART

It will be appreciated that in heath care practice and catering and food handling businesses, utmost care must be taken to prevent the transfer of infectious organisms from one person to the next or between animal subjects. Hence, the need to wash disinfect and/or sterilise devices used in health care, such as medical, dental and veterinary applications, as well as cooking and catering utensils, such as knives, forks, plates, pots and the like, is well known. A real problem experienced in the re-use of such devices is the adherence of pathogenic microorganisms and biofilm on surfaces of such devices. Biofilm refers to a conglomerate of microorganisms that are embedded in a structural matrix of macromolecules, such as exopolymers, wherein the matrix enables the colonizing cells to withstand normal treatment doses of biocides.

Methods such as scrubbing, boiling and steaming are often employed to destroy harmful pathogens and to disinfect and sterilise such devices. Also, the utilisation of disinfectants, sterilising agents and dispersants is common in such disinfecting and sterilising processes. These methods, however, are generally time consuming and costly and require suitable apparatus, sterilizing agents, effluent treatment processes and disposal facilities for sterilizing, disinfecting and dispersing chemicals and sterilizing agents.

In order to remove the soil, contamination, biofilm and/or residue on such devices, it is often necessary manually to scrub and/or ultrasonically treat soiled equipment as a washing phase prior to disinfecting and/or sterilizing the equipment in a subsequent disinfecting and/or sterilizing phase. The disadvantage of this typically two-phase process is that, in addition to some of the problems previously mentioned, it is time-consuming, costly and often impractical.

Existing apparatus includes, inter alia, electronic cleaners and baths, using conventional disinfectant solutions such as gluteraldehyde. However, these solutions often present further disadvantages due to their toxicity, resulting in dermatological as well as respiratory disorders. Autoclaves are also frequently used in cleaning health care devices, but due to the high temperatures at which these apparatus operate and the mode of operation, the devices are often damaged or destroyed in an autoclave.

In an effort to avoid cross-contamination between patients or animal subjects, disposable health care devices have been developed, which instead of being disinfected and/or sterilised, are discarded after a single use. However, it will be appreciated that not only is such practice often expensive, but it is often impossible or impractical to dispose of all health care devices that have been in contact with a patient after a single use.

It has long been known that electrolysis of fluids, for example saline solutions, results in the production of useful products, such as chlorine and ozone, which are especially useful as in-vitro microbicides for cleaning hard surfaces. So, for example, U.S. Pat. No. 5,462,644 discloses a method of sterilising and disinfecting equipment that are contaminated with biofilm by killing of the microorganisms in the biofilm, wherein the method includes the steps of suspending the contaminated equipment in a bath of electrically conductive electrolyte solution, and applying an electric field to the solution so as to kill the microorganisms. The electrolyte solution optionally may include an effective amount of a sterilant or disinfectant. A disadvantage associated with this method is that a suitable electric current must be applied to the bath continuously to effect working of the invention. In addition, the method causes substantial discomfort in patients when used in treating in-vivo infections.

U.S. Pat. No. 6,117,285 also discloses a system for sterilizing equipment, including medical and dental instruments. Particularly, the invention discloses an apparatus for producing an electrolysed fluid, such as a saline solution, that can be used for disinfecting and sterilising medical and dental equipment. More particularly, the apparatus comprises a container for holding a fluid to be electrolysed, power supply means to provide a source of electric current, and a first and second electrode immersed in the fluid and connected to the power supply means, the arrangement being such that the fluid is electrolysed as the current is passed there through. The invention also discloses a system for disinfecting and/or sterilising health care equipment that includes at least one conduit through the equipment, where the equipment are bathed in the electrolysed saline solution and where the system provides for through flow of electrolysed solution, through the conduit and over the surfaces of the equipment.

A disadvantage associated with this system is that the resultant electrolysed solution is produced in relatively small quantities on a batch or discontinuous basis. Further, the products produced at the anode and the cathode are intricately mixed so that the electrolysed solution comprises a mixture of anolyte and catholyte in a single solution. However, the respective effectiveness of the catholyte and anolyte is at least partially neutralised when they are produced and harvested as a single solution.

Electrolytically Activated Water and Treatment of Biofilm

The authors, in accordance with the requirements of this invention, utilised a cylindrical electrolytic device, having at least one electrolytic cell, in which the anodic and cathodic chambers are separated by a permeable membrane and the specific design of which permits the harnessing of two distinct, separate and electrochemically different product streams of activated water, in a process known as electrolytic activation (EA) or electrochemical activation (ECA).

The solutions remain active for a limited period. During this period of increased activity, these meta-stable solutions have been shown to have applications in a diverse array of technological processes, often as a substitute for traditional chemical agents. Irrespective of the characteristics of the specific solution, where activation status can extend from hours to days, the resultant meta-stable solutions following decay of the state of activation revert to benign water with the composition of the feed.

In addition, the ability to consistently produce two or more distinct, separate and electrochemically different product streams of activated water of specific quality as well as unique and proven attributes, on a demand driven basis, with no adverse environmental consequences, significantly differentiates the electrolytic technology applied in this invention from the electrolytic devices previously utilised or proposed for utilisation in, for example, the dental industry.

Principles of EA Technology in a Cylindrical Electrolytic Device

Water of varying mineralisation is passed through the cylindrical electrolytic cell, the specific design of which permits the production of two distinct and electrochemically different streams, electrolytically activated, low concentration saline solutions.

The design of the specific cylindrical cell utilised by the authors for this invention is such as to ensure a uniformly high voltage electrical field through which each micro-volume of water must pass. This electric field created in the cylindrical cell has a high potential gradient and results in the creation of solutions of which the pH, oxidation reduction potential (ORP) and other physico-chemical properties, lie outside of the range that can normally be achieved by conventional chemical or most electrolytic means.

Two separate streams of activated solutions are produced, namely anolyte and catholyte. Depending on the production methods used and conditions of operation of the device, the anolyte typically can have a pH range of 1.5 to 9 and an oxidation-reduction potential (ORP) of +150 mV to +1200 mV. The anolyte is oxidizing, due to the presence of a mixture of oxidising free radicals, and has an antimicrobial effect. The catholyte that is produced, typically can have a pH range of 8.5 to 13 and an ORP of about −150 mV to −900 mV. The catholyte has reducing and surfactant properties and is an antioxidant.

One of the advantages of the design of the specific cylindrical cell utilised by the authors for this invention is that the chemical composition of the two solutions can be altered by utilizing various hydraulic flow arrangements, linking electrolytic cell modules in various configurations in order optimally to address the requirements of specific areas of application. Some other variables are flow rate, hydraulic pressure, concentration, temperature, current density, and voltage on the electrodes.

Aside from its distinctive attributes, the negatively charged anti-oxidant solution, i.e. the catholyte, can also be channelled back into the anode chamber, thereby modulating the quality of the positively charged oxidant solution, i.e. the anolyte that is produced. Depending on the specifications of the required application, variations in the design of the hydraulic systems can be effected to meet the requisite objectives.

Properties of Electrolytically Activated Solutions

The properties of electrolytically activated solutions are dependent upon a number of factors. These factors comprise the solution flow rate through the cell, type of salt, the voltage and current being applied, temperature, inter-flow dynamics of the solutions between the anode and cathode chambers, such as the degree of feedback of catholyte into the anolyte chamber, the design and geometry of the cell and the degree of mineralisation of the water.

During the process of electrolytic activation in the electrolytic cell utilised by the authors, three broad classes of product are believed to be produced, namely:

(i) Stable products: These are acids (in the anolyte) and bases (in the catholyte) that influence the pH of the solution in question, as well as other active species;

(ii) Highly active unstable products: These include free radicals and other active ion species with a half-life of typically less than 48 hours. Included here are electrically and chemically active micro bubbles of electrolytic gas, 0.2 to 0.5 micrometer in diameter and with concentrations of up to 10⁷ ml⁻¹, distributed uniformly through the solution. All these species serve to enhance the ORP of the anolyte and catholyte;

(iii) Quasi-stable structures: These are structures formed at or near the electrode surface as a consequence of the very high voltage gradient (10⁶ V cm⁻¹) in those regions. These are free structural complexes of hydrated membranes around ions, molecules, radicals and atoms. The size of these water clusters is reduced from about 13-18 to approximately 5-6 molecules per cluster. All these features enhance the diffusion, catalytic and biocatalytic properties of the water.

It is important to note that the level of mineralisation of input water required to generate optimally metastable solutions is insignificantly different from the composition of potable water. However, the heightened electrical activity and altered physico-chemical attributes of the solutions differ significantly from the inactivated state, yet they remain non-toxic to mammalian tissue and the environment. Without maintenance of the activated state, these diverse products degrade to the relaxed state of benign water and the anomalous attributes of the activated solutions such as altered conductivity and surface tension similarly revert to pre-activation status.

Biocidal Properties of Anolyte and Mixed Anolyte and Catholyte

Most of the earlier technologies that have employed electrolytic activation to generate biocidal solutions have not been capable of separating the anolyte and catholyte solutions during generation in the cell. In these earlier technologies, the two opposing solutions have greatly neutralised each other with regard to potential electrical activity.

One of the advantages of the more modern ECA systems is that the biocidal activity of hypochlorous acid generated in these systems is up to 300 times more effective than the sodium hypochlorite generated by earlier systems. Additionally, comparison of neutral anolyte (pH=7) with alkaline gluteraldehyde (pH=8.5) showed that the latter required a concentration of 2% versus 0.05% of the former, in order to achieve the same biocidal efficacy. Similarly, it has been shown that a 5% solution of sodium hypochlorite (Jik) can only be used for purposes of disinfection, whilst a 0.03% solution of neutral anolyte has both disinfecting and sterilising properties. In general, the biocidal activity of non-activated neutral anolyte (only stable products and no electrical charge) is 80 times the potential activity of the hypochlorite solution, but still exhibits only one third of the full biocidal potential of the optimally activated ECA solution.

Thus, using non-toxic salts, these activated solutions have been shown conclusively to exceed chemically derived “equivalents” both in low dosage effectiveness as well as physico-chemical properties. This heightened biocidal capacity relative to traditional chemical solutions permits the incorporation of activated solutions at substantially lower dose rates, eliminating the risk of toxicity and adverse environmental impact, while providing cost effective resolutions.

Acidic Anolyte Solutions in Dental Units

The use of electrolytically activated low concentration saline solutions as biocides in dental unit water lines (DUWL) is proposed and disclosed in numerous documents, including international patent application PCT/US99/29013, published under WO 00/33757. This application, PCT/US99/29013 proposes the use of acidic electrolysed water having a pH of 2.5-6.5 in continuous contact with the interior surfaces of the DUWL's during operation of the dental appliances, both as biocide for the biofilm and as operating fluid for the dental appliances.

PCT/US99/29013 focuses on two types of electrolytic systems, both producing its acidic anolyte from a plate reactor-type, electrolytic cell, and proposes that it is incorporated into dental systems for disinfecting and reducing of bio-film in DUWL's. The first system makes use of a membrane to generate and separate distinct anolyte and catholyte solutions. This system generates very acidic anolyte at a pH 2-3,5. The second system does not use a membrane and generates only one stream of solution. PCT/US99/29013 proposes the addition of HCl (hydrochloric acid) into the feed of the second system, so as to increase the concentration of chloride ions and, in order to increase the microcidal efficacy of the anolyte, to lower the pH even further.

A material disadvantage of the acidic anolyte solutions proposed in PCT/US99/29013 is their toxicity, due to their relatively high chlorine and sodium hypochlorite content. In fact, it is believed that there is relatively little difference between the acid anolyte solutions as proposed and household bleach, with the latter being substantially simpler and cheaper to procure.

A further disadvantage of the acidic anolyte solutions proposed in PCT/US99/29013 is that they are advocated merely to reduce biofilm, and thus their apparent inability to eliminate biofilm, potentially allowing the DUWL's to develop resistant strains of biofilm, with the accompanying implication of serious health risks. More particularly, PCT/US99/29013 only proposes the disinfection of the DUWL's with reference to the cited microbial results, but does not propose the sterilisation of the DUWL's nor does it disclose any evidence of the removal of biofilm from the inner surfaces of the DUWL's. In fact, it is common knowledge that disinfection of water does not show/prove elimination or even reduction in biofilm.

In addition, PCT/US9929013 makes reference to the use of Japanese electrolyzers, which, as reported in a scientific paper published by Horiba et al in Oral Surgery, Oral Medicine, Oral Pathology, Volume 87, No.1, January 1999, proved ineffective against Bacillus subtilis, thus supporting the belief that the different electrolytic devices produce different solutions with levels of efficacy.

Further, and with reference to the adding of a dilute HCl solution to the electrolyzer to increase the chlorine concentration resulting in additional chlorine ions which increases the cleansing effect, it is believed that the acidic solutions without the added HCl is sub-optimally effective. It has been well documented that HCl, although a very effective biocide, has proven sub-optimal efficacy against biofilm. Thus, by adding HCl to the process water, one may improve the microcidal efficacy of the product to some extent but not the removal and elimination of the biofilm.

In addition, the relatively high concentrations of sodium hypochlorite generated result in the generation of relatively high levels of tri-halomethanes, thus increasing the carcinogenic potential of the solutions. PCT/US99/29013 thus proposes the use and incorporation of a sodium hypochlorite generator, which has contingent disadvantages and which defeats the whole purpose of using electrolytically activated saline solutions as biocides.

### OBJECT OF THE INVENTION

It is accordingly an object of the present invention to provide a relatively inexpensive, but effective method of and equipment for washing, disinfecting and/or sterilizing health care devices that will overcome or minimise the disadvantages experienced with known systems of this kind, or at least to provide a useful and economical alternative to known methods and systems.

### DISCLOSURE OF THE INVENTION

According to a first aspect of the invention there is provided a method for automatically washing, disinfecting and/or sterilizing health care equipment as well as cooking and catering utensils, the method including the steps of placing the equipment to be washed in an enclosure or on an appropriate conveyor mechanism; introducing a first electrochemically activated aqueous solution into the enclosure, the first solution being characterised therein that it has dispersing or surfactant characteristics for at least partially dispersing contamination, pathogenic microorganisms and/or a biofilm or, the like; and introducing a second electrochemically activated aqueous solution into the enclosure, the second solution being characterised therein that it has biocidal characteristics for killing microorganisms and disinfecting and/or sterilizing the equipment.

The method of the invention may be characterised therein that the electrochemically activated aqueous solutions are introduced into the enclosure in the form of a spray. For the purpose of this document, the term “spray” will be interpreted to include a fog, splatter, splash, mist, vapour, steam, aerosol or the like substantially particulate liquid matter or droplets. Preferably, but not exclusively, the spray may comprise of particulate liquid matter or droplets with an average size of less than 100 μm in diameter.

The first, second and subsequent electrochemically activated aqueous solutions may be introduced into the enclosure either sequentially or simultaneously. The method may include the steps of alternately or simultaneously introducing the first and second solutions in an application-specific sequence wherein the sequence of introduction of the solutions into the enclosure and the duration and conditions of contact are determined by the degree and nature of contamination or soiling in a particular application. The first and second solutions also may be introduced as a mixture comprising both the first and second electrochemically activated solutions, wherein the solutions may be mixed according to any preferred ratio, the arrangement being such that the first and second solutions and the mixture alternately or simultaneously may be introduced according to a predetermined application-specific sequence and protocol.

The aqueous solutions may be selected from a group consisting of anion-containing and cation-containing aqueous solution respectively. The anion-containing solution is referred to hereinafter for brevity as the “anolyte solution” or “anolyte” and the cation-containing solution is referred to herein for brevity as the “catholyte solution” or “catholyte”. Particularly, the first electrochemically activated aqueous solution is a catholyte having predominantly dispersing or surfactant characteristics, and whereas the second electrochemically activated aqueous solution is an anolyte having predominantly biocidal characteristics.

The anion-containing solution and the cation-containing solution may be produced by an electrochemical reactor or so-called electrolysis machine, comprising a through flow electrochemical cell having two co-axial cylindrical electrodes, and having a co-axial diaphragm or membrane between the two electrodes so as to separate an annular inter-electrode space into a catholytic and an anolytic chamber.

The electrochemically activated aqueous solutions may be prepared by means of electrolysis of an aqueous solution of a salt. The salt may be sodium chloride (NaCl) or potassium chloride (KCl). The salt also may be selected from a group including HCO3, CO3, SO4, NO3, PO4, any combination thereof or the like. The salt solution may be electrolysed to produce the anolyte and the catholyte with mixed oxidant and mixed reductant species. These species may be labile and after about 96 hours, the concentration and activity of the various activated species may reduce substantially with relatively little or no active residues being produced.

The microcidal solution for use in the method of the invention may be produced from an aqueous NaCl solution, the concentration of which may vary between 0,0001% to 1% and more specifically between 0.05% and 0.5% and preferably between 0.05% and 0.25%, electrolysed to produce radical cation and radical anion species.

The anolyte solution may have a redox potential of about +200 to +1100 mV and more specifically about +600 to +850 mV and preferably equal or more than +713 mV and a TDS of about 2-4 g/l. The anolyte solution may have a pH of about 6.75 to 8.5, preferably about 7.0 to 7.6, and a conductivity of about 0.1 to 10 mS/cm and more specifically of about 0.15 to 4.08 mS/cm, being produced at a current of about 5 to 7 Amperes, a voltage of approximately between 12V and 24V, thus providing a relatively high voltage gradient or electric field intensity at the interface between the electrode surface and electrolyte, estimated to be about 10⁶ V/cm, and a flow rate of about 50 to 500 ml/min and more specifically about 300 to 350 ml/min. The anolyte solution may include species such as ClO; ClO⁻; HClO; OH⁻; HO2⁻; H2O2; O3; S2O82⁻ and Cl₂O₆²⁻.

The above radicals in the anolyte solution have been found to have a suitable synergistic microbial effect against viral organisms, spore and cyst-forming bacteria, fungi and yeasts. The above anolyte has been found to have a suitable synergistic anti-microbial and/or anti-viral effect which compares favourably with sodium hypochlorite and have been found to be particularly effective against Prevotella intermedia, Porphyromonas gingivalis, Streptococcus mutans and Enterococcus faecalis.

The catholyte solution may have a pH of about 7.5 to 12.0 and a redox potential of about −150 to −950 mV and more particularly, about −850 mV and a conductivity of about 5.92 to 6.03 mS/cm. The catholyte solution may include species such as NaOH; KOH; Ca(OH)₂; Mg(OH)₂; HO⁻; H₃O₂; HO2⁻; H₂O₂⁻; O₂⁻; OH⁻; and O₂²⁻.

The inorganic components of both the anolyte and the catholyte solutions may include varying quantities of Al, Ca, Mg, Mn, K, Na, Mo, ammonium, orthophosphate, silica and chloride. The varying levels of saline concentration and the mineral content of he feed water, as well as the operational parameters of the electrochemical reactor, such as the different flow rates, flow regimes, flow paths and—rates of recycle, currents and potential differences, may be adjustable so as to produce anolyte and catholyte with suitable physical and chemical characteristics, with specific conductivity, redox potential and pH, concentration of “activated species”, and other characteristics, for particular applications.

It is believed that in addition to the normal mechanisms of action involved in elimination of micro-organisms, the oxidising free radicals and other constituents, such as micro-bubbles, present in the anolyte solution act synergistically at a bacterial cellular level, also killing the micro-organisms in an electrostatic manner.

Where used as a mixture, the efficacy of the mixed anolyte and catholyte solution may depend upon the concentration of the mixed anolyte and catholyte solution in the receiving water, as measured by the pH, amperage, oxidation-reduction potential (ORP), conductivity and TDS of the mixed anolyte and catholyte solution, the exposure time and the mixed anolyte and catholyte solution and the temperature during application.

Both the chemical and physical characteristics of the anolyte and the catholyte, preferably the redox potential, the pH, concentration and mixing ratio, as well as flow rate, pressure and temperature are adjustable so as to be suitable for washing, disinfecting, and/or sterilizing health care equipment and cooking and catering utensils for particular applications.

According to a second aspect of the invention there is provided apparatus for use in a method for automatically washing, disinfecting and/or sterilizing health care equipment and cooking and catering utensils, the apparatus including an electrochemical reactor or so-called electrolysis machine for producing first and second electrochemically activated aqueous solutions, the electrochemical reactor having a through flow electrochemical cell with two co-axial cylindrical electrodes, and having a co-axial diaphragm between the two electrodes so as to separate an annular inter-electrode space into a catholytic and an anolytic chamber; an enclosure for receiving and enclosing the equipment therein; and means for introducing the first, the second and subsequent electrochemically activated, aqueous solutions sequentially, alternatively simultaneously, into the enclosure.

According to a third aspect of the invention there is provided apparatus for automatically washing, disinfecting and/or sterilizing health care equipment and cooking and catering utensils, the apparatus comprising an enclosure for receiving and enclosing the equipment therein; and means for introducing, either sequentially or simultaneously, the first, second and any subsequent electrochemically activated aqueous solutions into the enclosure.

The apparatus may be characterised therein that the first and second electrochemically activated aqueous solutions are introduced into the enclosure in the form of a spray. Particularly, the anolyte and catholyte may be introduced as two distinct spray feeds. The catholyte and anolyte spray feeds may be introduced either simultaneously or sequentially. Alternatively, the catholyte and anolyte may be pre-harvested separately and then premixed in a preferred ratio for producing desired characteristics, before introducing the same into the enclosure as a premixed spray feed.

Alternatively, the first and second electrochemically activated aqueous solutions may be introduced into the enclosure as two distinct fluid feeds.

According to yet a further embodiment of the invention, the electrochemically activated aqueous solutions may be introduced sequentially into the enclosure first as a spray feed and then as a fluid feed. The spray feed either may comprise two distinct anolyte and catholyte spray feeds, or a single premixed spray feed comprising both anolyte and catholyte in solution. The fluid feed also either may comprise two distinct anolyte and catholyte fluid feeds, or a single premixed fluid feed comprising both anolyte and catholyte in solution.

The apparatus may include means for adjusting the physical and/or chemical characteristics of the electrochemically activated aqueous solutions, such as the redox potential and/or the pH and/or temperature and/or pressure and/or flow rate and/or flow configuration, so as to adjust the dispersing, disinfecting and/or sterilizing characteristics of the solutions for particular applications.

According to a fourth aspect of the invention there is provided a facility having apparatus for washing, disinfecting and/or sterilizing health care devices and/or cooking and catering utensils, the apparatus being substantially as hereinbefore defined.

### SPECIFIC EMBODIMENT OF THE INVENTION

An embodiment of the invention will now be described by means of a non-limiting example only and with reference to the accompanying drawings wherein

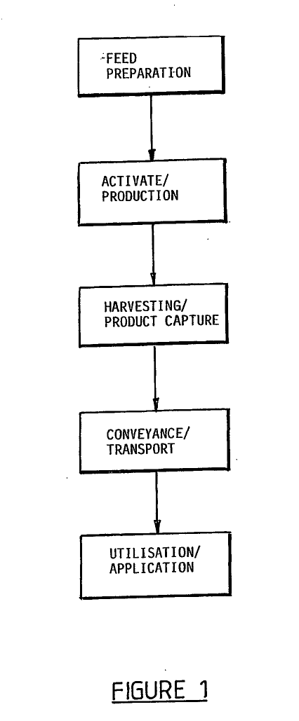

FIG. 1 is a flow chart of he method according to the invention; and

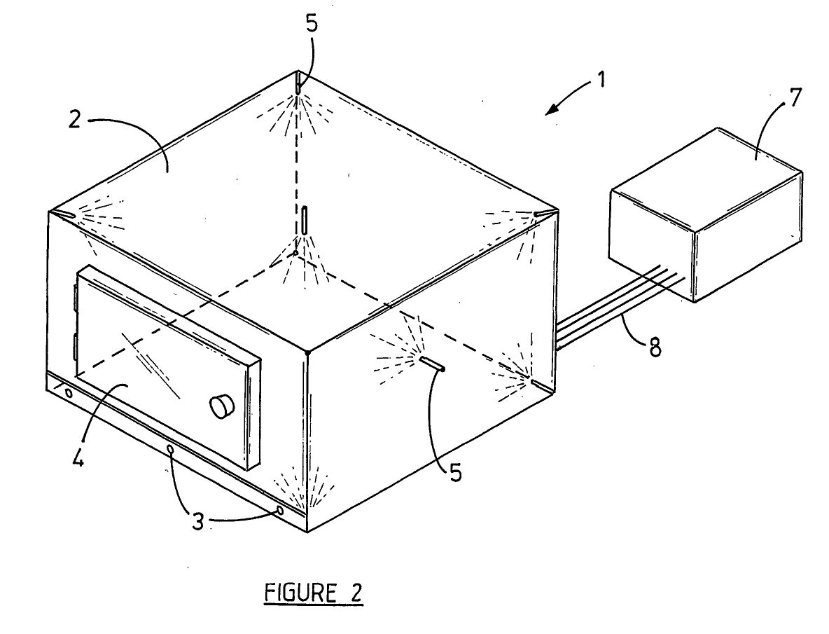

FIG. 2 is a diagrammatic illustration of an apparatus according to one embodiment of the invention.

The basic electrolytic cells used to generate the electrolytically activated solutions utilised in this specification are substantially as disclosed in U.S. Pat. No. 5,635,040. The cells are modular units, and, in various reactor configurations or devices, form the basis of the equipment disclosed in this specification, with the operational specifications for the reactors being optimised for each specific application. Particularly, the electrochemical reactor may be a so-called Flow-through Electrolytic Module (FEM) as also described by Bakhir in U.S. Pat. No. 5,427,667.

The cell includes a cylindrical metal vessel typically about 210 mm long×16 mm in diameter, having a central rod anode (positive electrode) located within a concentric ceramic tube membrane. The outer tubular wall of the cell reactor acts as the cathode (negative electrode). Provision is made for inlet and outlet ports for the passage of the fluid through it.

Effectively, the ceramic membrane divides the cell into two compartments, the anode compartment and the cathode compartment. Water enters the cell and exits from these compartments as two streams, namely the anolyte and the catholyte, respectively. If so desired, some or all of the catholyte may be returned to the anode compartment so as to vary the properties of the anolyte being produced. A number of other hydraulic system configurations also exist, all of which are designed to achieve specific objectives.

The design of the cell is such as to ensure a very high uniform electric field through which each micro volume of water must pass. In so doing the molecules of water in the anolyte and catholyte acquire special properties which cannot be reproduced by other (more conventional chemical) means. This electrolytic treatment results in the creation of anolyte and catholyte solutions whose pH, oxidation-reduction potentials (ORP) and other physico-chemical properties lie outside of the range that can be achieved by conventional chemical means.

Please note that the pH, oxidation-reduction (Redox) potential (ORP) and concentration values of chlorine, chlorides and other dissolved salts have been determined, unless otherwise stated, as per standard methods of examination of water and effluents.

Please note further that the annotation used for the various electrolytically generated solutions identified in this specification are as found in the Russian literature and patents of Bakhir et al and are as follows:

Anolyte:

1.1 A—Electrically Activated Acidic Anolyte

pH: <5,0

ORP: +800 . . .+1200 mV CSE

active species: C1₂, HClO, HCl, HO*₂

This solution results when there is no catholyte feedback and the mineralisation level is high (>5 g/l). Chlorine gas is evolved, the solution is highly oxidizing, corrosive and microcidal. The products are mostly stable.

1.2 AN—Electrically Activated Neutral pH Anolyte

pH: 5,0-7,0

ORP: +600 . . .+900 mV

active species: HClO, O₃, HO*, HO*₂

Here some catholyte is re-circulated to the anode compartment and the mineralisation is generally low (<3 g/l). Under these conditions, the formation of highly active but unstable species is favoured. The solution is microcidal but not corrosive, and harmless to human or animal tissue.

1.3 ANK—Electrically Activated Neutral pH Anolyte

pH: 7,2-8,2

ORP: +250-+800 mV

active species: HClO, CIO⁻, HO₂⁻, HO*₂, HO*, H₂O₂, ¹O₂, Cl⁻

Here a larger flow of catholyte is re-circulated resulting in a higher pH. The solution is still oxidizing and has similar properties to AN, but with a greater degree of short-term activation.

1.4 AND—Electrically Activated Neutral pH Anolyte

pH: 6,8-7,8

ORP: +700-+1100 mV

active species: HClO, CIO⁻, HO⁻₂, HO*₂, H₂O₂, ¹O₂, Cl*, HClO₂, C1O₂, O₃, HO*, O*

The solution has a rather high positive ORP and can be used for disinfection.

2. Catholyte:

2.1 K—Electrically Activated Alkaline Catholyte

pH: >9,0

ORP: −700-−820 mV

active species: NaOH, O⁻₂, HO*₂, HO₋₂, OH⁻, OH*, HO₂⁻, O²⁻₂

This solution usually has a pH of 11-12 and is highly reducing. It is very active but the relaxation times are significantly shorter than for anolyte solutions.

2.2 KN—Electrically Activated Neutral Catholyte

pH: <9,0

ORP: −300-−500 mV

active species: O⁻₂, HO*₂, HO⁻₂, H₂O₂, H*, OH*

When mixed together, post production and extrinsically to the generating device, in the “as produced” ratios, the anolyte and catholyte form a unique solution, which has, both microcidal as well as surfactant properties. The capacity of a single solution possessed of both these attributes concurrently cannot be replicated with currently available chemical formulations. The dual attributes of this mixture have also been shown to be non-toxic for human tissue, as well as having a low corrosion potential profile. The mixture, with its strong oxidation-reduction potential has the capacity to effect the necessary electron transfer between the metastable radical species of the solution and the specific electrical charges present on the biofilm surface, thus destabilising the electrolytic forces at the interface of the gluco-calyx matrix (GCM) and the exposed (non-biofilm coated) conduit surface. This results in the reduced adherence and hence dislodging of the biofilm matrix.

Non-Limiting Example of Current Invention

The current invention relates to apparatus (1) for use in a method for automatically washing, disinfecting and sterilizing health care equipment, as well as cooking and catering utensils (not shown). The apparatus (1) includes an electrochemical reactor or so-called electrolysis machine (7), having a through flow electrochemical cell with two co-axial cylindrical electrodes, with a co-axial diaphragm between them so as to separate an annular inter-electrode space into a catalytic and an analytic chamber. The apparatus also includes an enclosure (2) for receiving and enclosing the equipment therein. The enclosure (2) and the electrochemical reactor (7) are connected to each other by intermediate electric and/or hydraulic connections (8). It will, however, be appreciated that the electrochemical reactor (7) also may integrally be formed with the enclosure (2).

The apparatus (1) further includes introduction means (5) for introducing the electrochemically activated aqueous solutions sequentially, alternatively simultaneously, into the enclosure (2).

The enclosure (2) is provided with a suitable closure means (4) and adjusting means (3) for adjusting the apparatus (1) so as to provide the required cycles of the first, second and any subsequent electrochemically activated aqueous solutions.

In use, the apparatus (1) is adjusted by adjusting the adjusting means (3) to the required cycles whereafter the equipment to be disinfected are enclosed in the enclosure (2).

The first electrochemically activated aqueous solution, in the form of a catholyte and the second electrochemically activated aqueous solution, in the form of an anolyte, are then sequentially introduced to first wash the equipment and then to disinfect and sterilize the same. However, as is clear from the invention, it is envisaged that the anolyte and the catholyte could be introduced simultaneously so as to wash, disinfect and sterilize the equipment to be disinfected in a single cycle.

The applicant believes that by introducing the catholyte and anolyte as two distinct feed streams, the effectiveness of the respective dispersing and microcidal characteristics of the catholyte and the anolyte is optimised. In addition, the initial introduction of the anolyte and catholyte as a spray, as opposed to a liquid, in certain applications has proven to be advantageous over the introduction of an electrolysed solution as a stream.

It if further envisaged that, in the application for cooking and catering utensils, the method of the invention could be used together with known detergents. It is believed, however, that the method of the invention will reduce the consumption of such detergents and the pollution potential of the effluent, as compared with using conventional detergents only.

It will be appreciated that many variations in detail are possible without departing from the scope or spirit of the invention as defined in the claims.

1. A method for automatically washing, disinfecting and/or sterilizing health care devices as well as cooking and catering utensils, the method including the steps of placing the equipment to be washed in an enclosure or on appropriate conveying means; introducing a first electrochemically activated aqueous solution into the enclosure, the first solution being characterised therein that it has dispersing or surfactant characteristics for at least partially dispersing contamination, pathogenic micro-organisms and/or biofilm, or the like; and introducing a second electrochemically activated aqueous solution into the enclosure, the second solution being characterised therein that it has biocidal characteristics for killing microorganisms and disinfecting and/or sterilizing the equipment.

2. The method according to claim 1 characterised therein that the electrochemically activated aqueous solutions are introduced into the enclosure in the form of a spray.

3. The method according to claim 2 characterised therein that the spray preferably, but not exclusively, comprise of particulate liquid matter or droplets with an average size of less than 100 μm in diameter.

4. The method according to claim 1 characterised therein that the first, second and any subsequent electrochemically activated aqueous solutions are introduced into the enclosure either sequentially or simultaneously.

5. The method according to claim 4 characterised therein that the first, second and any subsequent electrochemically activated aqueous solutions are introduced either alternately or simultaneously in an application-specific sequence wherein the sequence and protocol of introduction of the solutions into the enclosure is determined by the degree and nature of contamination or soiling in a particular application.

6. The method according to claims 4 or 5 characterised therein that the first and second solutions also may be introduced as a mixture comprising both the first and second electrochemically activated solutions, wherein the solutions may be premixed according to any preferred ratio, the arrangement being such that the first and second solutions and the mixture alternately or simultaneously may be introduced according to a predetermined application-specific sequence.

7. The method according to claim 1 characterised therein that the first, second and any subsequent aqueous solutions are selected from a group consisting of anion-containing and cation-containing aqueous solutions respectively.

8. The method according to claim 7 characterised therein that the first electrochemically activated aqueous solution is a catholyte having predominantly dispersing or surfactant characteristics, and whereas the second electrochemically activated aqueous solution is an anolyte having predominantly biocidal characteristics.

9. The method according to claim 7 characterised therein that the anion-containing solution and the cation-containing solution are produced by an electrochemical reactor or so-called electrolysis machine, comprising a through flow electrochemical cell having two co-axial cylindrical electrodes, and having a co-axial diaphragm or membrane between the two electrodes so as to separate an annular inter-electrode space into a catholytic and an anolytic chamber.

10. The method according to claim 1 characterised therein that the electrochemically activated aqueous solutions are prepared by means of electrolysis of an aqueous solution of a salt.

11. The method according to claim 10 characterised therein that the salt is sodium chloride (NaCl) or potassium chloride (KCl).

12. The method according to claim 10 characterised therein that the salt is selected from a group including HCO3, CO3, SO4, NO3, PO4, any combination thereof or the like.

13. The method according to claims 10 and 11 characterised therein that the electrochemically activated aqueous solutions are prepared by means of electrolysis of an aqueous NaCl solution, the concentration of which varies between 0,0001% to 1% and more specifically between 0.05% and 0.5% and preferably between 0.05% and 0.25%, electrolysed to produce radical cation and radical anion species.

14. The method according to claim 8 characterised therein that the anolyte solution has a redox potential of about +200 to +1100 mV and more specifically about +600 to +850 mV and preferably equal or more than +713 mV and a TDS of about 2-4 g/l.

15. The method according to claims 8 and 14 characterised therein that the anolyte solution has a pH of about 6.75 to 8.5, preferably about 7.0 to 7.6, and a conductivity of about 0.1 to 10 mS/cm and more specifically of about 0.15 to 4.08 mS/cm, being produced at a current of about 5 to 7 Amperes, a voltage of approximately between 12V and 24V, and a flow rate of about 50 to 500 ml/min and more specifically about 300 to 350 ml/min.

16. The method according to claim 8 characterised therein that the anolyte solution includes species such as ClO; ClO⁻; HClO; OH⁻;. HO₂⁻; H₂O₂; O₃; S₂O₈²⁻; and Cl₂O₆²⁻.

17. The method according to claim 8 characterised therein that the catholyte solution has a pH of about 7.5 to 12.0 and a redox potential of about −150 to −950 mV, and more specifically about −850 mV and a conductivity of about 5.92 to 6.03 mS/cm.

18. The method according to claim 8 characterised therein that the catholyte solution includes species such as NaOH; KOH; Ca(OH)₂; Mg(OH)₂; HO⁻; H₃O₂; HO₂⁻; H₂O₂⁻; O₂⁻; OH⁻; and O₂²⁻.

19. The method according to claim 8 characterised therein that the chemical and physical characteristics of both the anolyte and the catholyte, such as the redox potential, the pH, concentration and mixing ratio, as well as flow rate, flow configuration, pressure and temperature, are adjustable so as to be suitable for washing, disinfecting and/or sterilizing, health care equipment and cooking and catering utensils for particular applications.

20. Apparatus for use in a method for automatically washing, disinfecting and/or sterilizing health care equipment and/or cooking and catering utensils, the apparatus including an electrochemical reactor or so-called electrolysis machine for producing first and second electrochemically activated aqueous solutions, the electrochemical reactor having a through flow electrochemical cell with two co-axial cylindrical electrodes, and having a co-axial diaphragm or membrane between the two electrodes so as to separate an annular inter-electrode space into a catholytic and an anolytic chamber; an enclosure for receiving and enclosing the equipment therein; and means for introducing the first, the second and any subsequent electrochemically activated aqueous solutions sequentially, alternatively simultaneously, into the enclosure.

21. Apparatus for automatically washing, disinfecting and/or sterilizing health care equipment and/or cooking and catering utensils, the apparatus comprising an enclosure for receiving and enclosing the equipment therein; and means for introducing, either sequentially or simultaneously, the first, second and any subsequent electrochemically activated aqueous solutions into the enclosure.

22. The apparatus according to either one of claims 20 or 21 characterised therein that the electrochemically activated aqueous solutions are introduced into the enclosure in the form of a spray.

23. The apparatus according to claim 22 characterised therein that the first electrochemically activated aqueous solution is a catholyte having predominantly dispersing or surfactant characteristics, whereas the second electrochemically activated aqueous solution is an anolyte having predominantly biocidal characteristics.

24. The apparatus according to claim 22 characterised therein that the anolyte and catholyte are introduced as two distinct spray feeds.

25. The apparatus according to claim 24 characterised therein that the catholyte and anolyte spray feeds are introduced either simultaneously or sequentially.

26. The apparatus according to claim 22 characterised therein that the catholyte and anolyte are pre-harvested separately and then premixed in a preferred ratio for producing a mixture with prerequisite characteristics, before introducing the same into the enclosure as a premixed spray feed.

27. The apparatus according to claim 20 or 21 characterised therein that the first and second electrochemically activated aqueous solutions are introduced into the enclosure as two distinct fluid feeds.

28. The apparatus according to claim 20 or 21 characterised therein that the first and second electrochemically activated aqueous solutions are introduced sequentially into the enclosure first as a spray feed and then as a fluid feed.

29. The apparatus according to claim 28 characterised therein that the spray feed either comprises two distinct anolyte and catholyte spray feeds, or a single premixed spray feed comprising both anolyte and catholyte in solution, and characterised therein that the fluid feed also either comprises two distinct anolyte and catholyte fluid feeds, or a single premixed fluid feed comprising both anolyte and catholyte in solution.

30. The apparatus according to claim 20 characterised therein that the apparatus includes means for adjusting the physical and/or chemical characteristics of the electrochemically activated aqueous solutions, such as the redox potential and/or the pH and/or temperature and/or pressure and/or flow rate, so as to adjust the dispensing, disinfecting and/or sterilizing characteristics of the solutions for particular applications.

31. A health care facility having apparatus for washing, disinfecting and/or sterilizing health care devices and/or cooking and catering utensils, the apparatus being substantially as hereinbefore defined.

32. The method according to claim 1 characterised therein that it provides for the continuous washing, disinfecting and/or sterilizing of health care equipment or cooking and catering utensils by continuously producing the electrochemically activated aqueous solutions and introducing the same into the enclosure.
